(19) United States
(12) Patent Application Publication
(10) Pub. No.: US 2014/0088409 A1
(43) Pub. Date: Mar. 27, 2014

(54) Receiving Coils for Magnetic Resonance Systems

(71) Applicants: Wen Ming Li, Shenzhen (CN); Jia Heng Tan, Shenzhen (CN); Tong Tong, Shenzhen (CN); Ying Lun Wang, Shenzhen (CN)

(72) Inventors: Wen Ming Li, Shenzhen (CN); Jia Heng Tan, Shenzhen (CN); Tong Tong, Shenzhen (CN); Ying Lun Wang, Shenzhen (CN)

(21) Appl. No.: 14/034,046

(22) Filed: Sep. 23, 2013

(30) Foreign Application Priority Data
Sep. 25, 2012 (CN) CN 201220490808.9

(51) Int. Cl. G01R 33/34; A61B 5/055

(52) U.S. Cl. CPC G01R 33/34; A61B 5/055
USPC 600/422; 324/322

(57) ABSTRACT

A receiving coil for a magnetic resonance system includes an antenna unit and a locating unit. The locating unit is used for locating a subject to be examined and is positioned inside the antenna unit. The receiving coil for a magnetic resonance system may improve a patient's level of comfort, reduce the complexity of operation, and improve the coil's signal-to-noise ratio.

### RELATED APPLICATIONS

This application claims the benefit of Chinese Patent Application No. CN 201220490808.9, filed Sep. 25, 2012, the entire contents of which are hereby incorporated herein by reference.

### TECHNICAL FIELD

The present teachings relate generally to magnetic resonance imaging and, in some embodiments, to receiving coils for magnetic resonance systems.

### BACKGROUND

A magnetic resonance imaging (MRI) system includes various coils, such as a body coil that covers a whole body area, a receiving coil that covers only a part of a body, and the like. The body coil is a self-transmitting and self-receiving coil. Since the coverage area of the body coil is relatively large, a higher transmitting power may be needed. The signal-to-noise ratio of an obtained image is relatively low, and the signal-to-noise ratio throughout the image is non-uniform. By contrast, since the coverage area of the receiving coil is relatively small (e.g., a knee area covered by a knee coil, a head covered by a head coil, a wrist covered by a wrist coil, etc.), the receiving coil receives only limited radio-frequency signals within the radio-frequency excitation range. The radio-frequency signals received by the coil are referred to as magnetic resonance signals, and are distinguished from radio-frequency signals of the transmission stage. The signal-to-noise ratio of an obtained image is relatively high. Moreover, the signal-to-noise ratio throughout the image is relatively uniform.

Magnetic resonance systems may be used to assist in stereotactic surgeries. For example, the location information about a subject to be examined and the location parameters of the subject with respect to reference coordinates are acquired using a receiving coil.

The receiving coil or a TR/RX coil of a magnetic resonance system may be used in assisting surgeries. By way of example, in the case of head surgery, a head coil is first secured to a patient bed. Next, a special frame (e.g., a locating unit) equipped with reference coordinates is worn on the head of a patient who is lying on the patient bed. Finally, the head wearing the locating frame is moved into the head coil. If the head coil includes both an upper part and a lower part, then the upper part will be closed. The placement of and adjustments to the locating frame may cause discomfort in the patient. In addition, the heaviness of the locating frame worn on the head of the patient may be a source of discomfort. Moreover, in using the locating unit, the head coil may reserve a redundancy space. As a result, the head coils may have a large volume resulting in a low signal-to-noise ratio.

### SUMMARY AND DESCRIPTION

The scope of the present invention is defined solely by the appended claims, and is not affected to any degree by the statements within this summary.

The present embodiments may obviate one or more of the drawbacks or limitations in the related art. For example, in some embodiments, a patient's level of comfort may be improved, the complexity of operation may be reduced, and/or the coil's signal-to-noise ratio may be improved.

In order to improve a patient's comfort level, reduce the complexity of operation, and improve the coil's signal-to-noise ratio, a receiving coil for a magnetic resonance system may be used that includes an antenna unit. The receiving coil further includes a locating unit that is used for locating a subject to be examined. The locating unit is positioned inside the antenna unit.

In some embodiments, the antenna unit and the locating unit are integrally formed.

In some embodiments, the receiving coil further includes a plurality of fastening units. The plurality of fastening units is used for connecting the locating unit to the antenna unit.

In some embodiments, the fastening unit includes a plurality of rivets and a plurality of fastening components. The rivets and the fastening components match each other.

In some embodiments, the plurality of rivets is located in the antenna unit, and the plurality of fastening components is located in the locating unit. In other embodiments, the plurality of rivets is located in the locating unit, and the plurality of fastening components is located in the antenna unit.

In some embodiments, the receiving coil further includes a sheet-like C-shaped arm. The sheet-like C-shaped arm is connected to one end of the antenna unit. In some embodiments, the plurality of rivets is located on the C-shaped arm, and the plurality of fastening components is located in the locating unit. In other embodiments, the plurality of rivets is located in the locating unit, and the plurality of fastening components is located on the C-shaped arm.

In some embodiments, the fastening component is a hook-shaped washer.

In some embodiments, the end of the fastening component that matches the rivet may be rotated about the other end of the fastening component.

In some embodiments, the locating unit includes three-dimensional orthogonal rulers. The rulers are configured to provide visible reference coordinates in a magnetic resonance image and are located outside the locating unit.

In some embodiments, the locating unit includes a supporting component. The supporting component is used for securing the subject to be examined and is located inside the locating unit.

In some embodiments, a receiving coil for a magnetic resonance system may improve a patient's level of comfort, reduce the complexity of operation, and improve the coil's signal-to-noise ratio.

### BRIEF DESCRIPTION OF THE DRAWINGS

FIG. 1 is a schematic diagram of an exemplary head coil for a magnetic resonance system in accordance with the present teachings.

FIG. 2 is a schematic diagram of an exemplary antenna unit and an exemplary fastening unit of a receiving coil for a magnetic resonance system in accordance with the present teachings.

FIG. 3 is a schematic diagram of an exemplary locating unit of a receiving coil for a magnetic resonance system in accordance with the present teachings.

### DETAILED DESCRIPTION

A receiving coil for a magnetic resonance system is described herein that, in some embodiments, may be used to improve a patient's comfort level, reduce the complexity of operation, and improve the coil's signal-to-noise ratio. In some embodiments, the receiving coil for a magnetic resonance system is provided for use in a stereotactic surgery of the head. In some embodiments, the head coil may be integrated internally with a locating unit. Moreover, in some embodiments, the head coil includes a signal receiving coil and a head-locating unit.

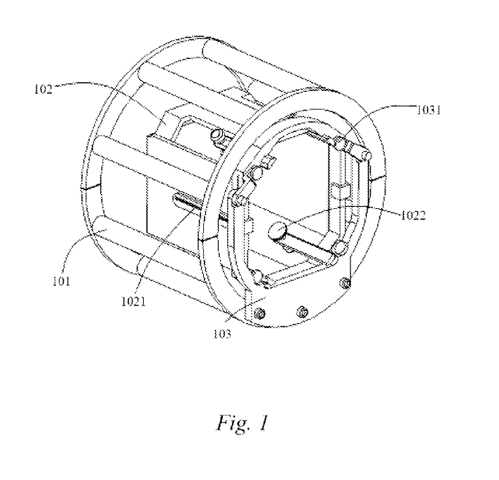

FIG. 1 is a schematic diagram of a receiving coil for a magnetic resonance system. As shown in FIG. 1, the head coil 100 for a magnetic resonance system includes an antenna unit 101, a locating unit 102, and a fastening unit 103.

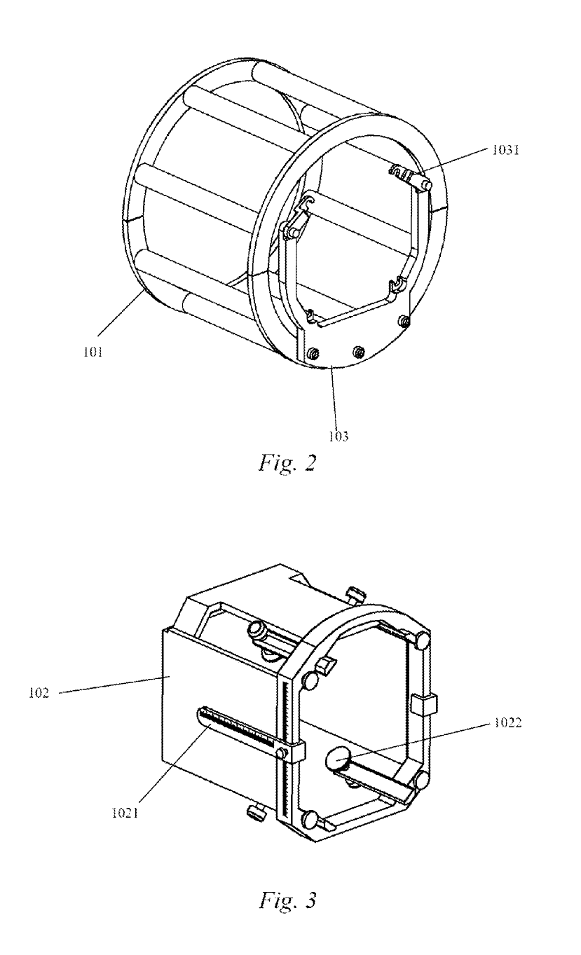

FIG. 2 is a schematic diagram of an antenna unit and a fastening unit of a receiving coil for a magnetic resonance system. As shown in FIGS. 1 and 2, the antenna unit 101 is a hollow cylinder configured for sending radio frequency signals to the head of a subject to be examined and/or for receiving radio frequency signals from the head of the subject to be examined, thereby generating a magnetic resonance image.

FIG. 3 is a schematic diagram of a locating unit of a receiving coil for a magnetic resonance system. As shown in FIGS. 1 and 3, the locating unit 102 is a hollow cylinder located inside the antenna unit 101. The locating unit 102 is used for locating the subject to be examined. In some embodiments, the locating unit 102 provides reference coordinates to the magnetic resonance image of the head of the subject to be examined. The locating unit 102 provides location parameters to the head of the subject to be examined during the stereotactic surgery. The locating unit 102 further includes rulers 1021 for displaying in the head magnetic resonance image of the subject to be examined. The rulers 1021 are configured to provide the location parameters for locating each detail of the head. For example, the rulers 1021 provide three-dimensional reference coordinates (e.g., length, width, and height). The rulers 1021 are located on the left side (or the right side) and the upside (or the downside) of the exterior of the locating unit 102. As shown in FIGS. 1 and 3, the ruler 1021 marking the reference coordinates of the length and height is located on the left side of the locating unit. The ruler 1021 marking the reference coordinate of the width is located on the downside of the locating unit (not shown). In some embodiments, the locating unit includes a supporting component 1022 configured for securing the head of the subject to be examined in the head coil 100. The supporting component 1022 is located inside the locating unit.

The locating unit 102 further includes four rivets. The rivets are located, respectively, in the four corners of the rectangular bottom of the locating unit. The rivets match the corresponding components of the fastening unit 103. The locating unit 102 and the fastening unit 103 may be integrally fixed by screwing or attaching the rivets to the corresponding components of the fastening unit 103. The connection modes of the locating unit 102 and the fastening unit 103 may include other connection modes than the rivet, including but not limited to fasteners.

FIG. 2 is a schematic diagram of an antenna unit and a fastening unit of a receiving coil for a magnetic resonance system. As shown in FIGS. 1 and 2, the fastening unit 103 is a sheet-like C-shaped arm. The fastening unit 103 is installed on one end of the antenna unit for attaching the locating unit 102 to the antenna unit 101. The fastening unit 103 and the antenna unit 101 may be integrally formed or fixed together through screws.

In some embodiments, the fastening unit includes four fastening components 1031. The four fastening components 1031 may be hook-shaped washers. Each two of the hook-shaped washers may be symmetrical and located, respectively, on the two arms of the C-shaped arm. The angle between the fastening component 1031 and the C-shaped arm is adjustable. The four rivets of the locating unit pass through the four fastening components 1031. Just by screwing the rivets, the locating unit 102 may be detachably fixed to the fastening unit 103 and the antenna unit 101.

As shown in FIGS. 1 and 2, the fastening component 1031 is a washer with one end thereof being hook-shaped. A rivet passes through the inner edge of the hook-shaped end of the washer and screws down that portion. The other end of the fastening component 1031 connects to the body of the fastening unit at a connection point. The end of the fastening component 1031 that matches the rivet is configured for rotating about the other end of the fastening component 1031. The angle between the fastening component 1031 and the C-shaped arm is adjustable to accommodate locating units 102 of various models and sizes.

In addition to the cylindrical antenna unit 101 shown in FIG. 2, the birdcage-like (e.g., metamorphic columnar) antenna unit may also be used. The birdcage-like antenna unit may optimize the structure of a coil according to head shape, and improve the signal-to-noise ratio of a head magnetic resonance image. In some embodiments, an antenna unit detachably composed of two or more components may also be used.

In some embodiments, the receiving coil for a magnetic resonance system may improve the quality of an image and provide more accurate location information about a subject to be examined, thereby assisting doctors in the performance of surgeries. Since, in some embodiments, the receiving coil for a magnetic resonance system and the locating unit are integrally formed, the volume of the apparatus is made smaller and, in turn, patient comfort may be increased.

A receiving coil for a magnetic resonance system is provided that includes an antenna unit and a locating unit. The locating unit is used for locating a subject to be examined and is positioned inside the antenna unit. In some embodiments, the receiving coil for the magnetic resonance system may improve a patient's comfort level, reduce the complexity of operation, and improve the coil's signal-to-noise ratio.

While the present invention has been described above by reference to various embodiments, it should be understood that many changes and modifications may be made to the described embodiments. It is therefore intended that the foregoing description be regarded as illustrative rather than limiting, and that it be understood that all equivalents and/or combinations of embodiments are intended to be included in this description.

It is to be understood that the elements and features recited in the appended claims may be combined in different ways to produce new claims that likewise fall within the scope of the present invention. Thus, whereas the dependent claims appended below depend from only a single independent or dependent claim, it is to be understood that these dependent claims may, alternatively, be made to depend in the alternative from any preceding claim—whether independent or dependent—and that such new combinations are to be understood as forming a part of the present specification.

1. A receiving coil for a magnetic resonance system, the receiving coil comprising:
an antenna unit; and
a locating unit positioned inside the antenna unit;
wherein the locating unit is configured for locating a subject to be examined.

2. The receiving coil of claim 1, wherein the antenna unit and the locating unit are integrally formed.

3. The receiving coil of claim 1, further comprising a fastening unit configured for connecting the locating unit to the antenna unit.

4. The receiving coil of claim 3, wherein the fastening unit comprises a rivet and a complementary fastening component.

5. The receiving coil of claim 4, wherein the rivet is located in the antenna unit, and wherein the fastening component is located in the locating unit.

6. The receiving coil of claim 4, wherein the rivet is located in the locating unit, and wherein the fastening component is located in the antenna unit.

7. The receiving coil of claim 4, further comprising a sheet-like C-shaped arm, wherein the sheet-like C-shaped arm is connected to one end of the antenna unit, wherein the rivet is located on the C-shaped arm, and wherein the fastening component is located in the locating unit.

8. The receiving coil of claim 4, further comprising a sheet-like C-shaped arm, wherein the sheet-like C-shaped arm is connected to one end of the antenna unit, wherein the rivet is located in the locating unit, and wherein the fastening component is located on the C-shaped arm.

9. The receiving coil of claim 4, wherein the fastening component comprises a hook-shaped washer.

10. The receiving coil of claim 5, wherein the fastening component comprises a hook-shaped washer.

11. The receiving coil of claim 6, wherein the fastening component comprises a hook-shaped washer.

12. The receiving coil of claim 7, wherein the fastening component comprises a hook-shaped washer.

13. The receiving coil of claim 8, wherein the fastening component comprises a hook-shaped washer.

14. The receiving coil of claim 4, wherein the fastening component has a first end that is complementary to the rivet and a second end, wherein the first end is configured for rotation about the second end.

15. The receiving coil of claim 5, wherein the fastening component has a first end that is complementary to the rivet and a second end, wherein the first end is configured for rotation about the second end.

16. The receiving coil of claim 6, wherein the fastening component has a first end that is complementary to the rivet and a second end, wherein the first end is configured for rotation about the second end.

17. The receiving coil of claim 7, wherein the fastening component has a first end that is complementary to the rivet and a second end, wherein the first end is configured for rotation about the second end.

18. The receiving coil of claim 8, wherein the fastening component has a first end that is complementary to the rivet and a second end, wherein the first end is configured for rotation about the second end.

19. The receiving coil of claim 1, wherein the locating unit comprises a three-dimensional orthogonal ruler, wherein the three-dimensional orthogonal ruler is located outside the locating unit and is configured to provide visible reference coordinates in a magnetic resonance image.

20. The receiving coil of claim 1, wherein the locating unit comprises a supporting component, and wherein the supporting component is located inside the locating unit and is configured for securing the subject to be examined.
